SEQUENCE LISTING (2 sequences)

SEQ ID NO: 1 - protein, 167 aa, Escherichia coli [sequence omitted]

SEQ ID NO: 2 - DNA, 504 bp, Escherichia coli
atgagtaccgctaaattagttaaatcaaaagcgaccaatctgctttatacccgcaacgatgtctccgacagcgagaaaaaagcaacagtagagttgctgaatcgccaggttatccagtttattgatctttctttgattaccaaacaagcgcactggaacatgcgcggcgctaacttcattgccgtacatgaaatgctggatggcttccgcaccgcactgatcgatcatctggataccatggcagaacgtgcagtgcagctgggcggtgtagctctggggaccactcaagttatcaacagcaaaaccccgctgaaaagttacccgctggacatccacaacgttcaggatcacctgaaagaactggctgaccgttacgcaatcgtcgctaatgacgtacgcaaagcgattggcgaagcgaaagatgacgacaccgcagatatcctgaccgccgcgtctcgcgacctggataaattcctgtggtttatcgagtctaacatcgaataa

(19) United States
(12) Patent Application Publication
(10) Pub. No.: US 2022/0251538 A1
(43) Pub. Date: Aug. 11, 2022

(54) METHOD FOR REMOVAL OF NUCLEIC ACIDS IMPURITIES FROM LIQUID COMPOSITION COMPRISING GENETICALLY ENGINEERED PARTICLES OR PROTEINS

(71) Applicants: Matjaz Vogelsang, Celje (SI); David Dobnik, Ljubljana (SI); Maja Ravnikar, Ljubljana (SI); Dominik Gaser, Zelezniki (SI)

(72) Inventors: Matjaz Vogelsang, Celje (SI); David Dobnik, Ljubljana (SI); Maja Ravnikar, Ljubljana (SI); Dominik Gaser, Zelezniki (SI)

(21) Appl. No.: 17/613,521

(22) PCT Filed: May 23, 2019
(86) PCT No.: PCT/IB2019/054310

§ 371 Date: Nov. 23, 2021

(51) Int. Cl. C12N 15/10; B03C 1/01; B01D 21/26

(52) U.S. Cl. CPC C12N 15/1013; B03C 2201/18; B01D 21/262; B03C 1/01

(57) ABSTRACT

The present disclosure provides improved methods for purifying recombinant protein, vaccine and gene therapy preparations, such as vectors in a suspension, as well as new means to better assay residual nucleic acids in a composition comprising genetically engineered particles. One aspect of the present disclosure is a method for purifying a liquid composition comprising genetically engineered particles from nucleic acid impurities comprising the steps of (i) adding a Dps protein to the suspension comprising genetically engineered particles, (ii) precipitating a complex comprising the nucleic acid impurities and the Dps protein and (iii) removing the precipitated complex comprising the nucleic acid impurities and Dps protein from the suspension comprising genetically engineered particles. Another aspect of the disclosure is a method to assay nucleic acid impurities in a liquid composition comprising genetically engineered particles or a pharmaceutical composition comprising genetically engineered particles.

### SEQUENCE LISTING

The instant application contains a Sequence Listing which has been submitted electronically in ASCII format and is hereby incorporated by reference in its entirety. Said ASCII copy, created on May 22, 2019, is named PCT003_2019_ST25.txt and is 2,492 bytes in size.

### FIELD OF THE DISCLOSURE

The present disclosure relates to the field of separating nucleic acids from a liquid composition, particularly from a composition comprising particles for use to deliver nucleic acids to a subject, or particles that require nucleic acids in a process for their preparation, such as gene therapy delivery systems or vaccines. The present disclosure relates particularly to purification of cell vectors, viral vectors and vaccines. The present disclosure relates to a method for purifying a liquid composition comprising genetically engineered particles from nucleic acid impurities, a mixture comprising genetically engineered particles and complexed nucleic acids, a pharmaceutical composition obtained by the method of purifying composition comprising genetically engineered particles, a method to assay residual nucleic acid impurities comprised in a liquid composition comprising genetically engineered particles or a pharmaceutical composition comprising genetically engineered particles, and to a new use of a DNA-binding protein from starved cells (Dps) protein.

### BACKGROUND OF THE DISCLOSURE

Vaccination is widely used public health measure for preventing or treating existing, recurrent or sporadically occurring diseases. On the other hand, gene therapy, where genetic material is transferred to a subject such as a patient to treat a disease, is gaining on importance. Gene therapy can be classified according to the class of disease, according to the gene delivery vehicle, and according to whether the vector is administered in vivo (directly into the patient) or ex vivo (in cultured cells taken from the patient that are subsequently transplanted back). Since 1990 gene therapy has been tested in clinical trials with variable results in cancer, monogenic diseases, infectious diseases, cardiovascular diseases, neurological diseases, ocular diseases and inflammatory diseases and other settings (Ginn SL et al. J. Gene Med., 2018, 20:e3015). More specifically, genetic diseases investigated include for example alpha-1-antitryspin deficiency, Batten's disease, Canavan's disease, cystic fibrosis, hemophilia B, Leber's congenital amaurosis (LCA2), lipoprotein lipase deficiency, Pompe's disease, Duchenne and limb girdle muscular dystrophies, and acquired disease, including Alzheimer's disease, heart failure, Parkinson's disease, rheumatoid arthritis and age-related macular degeneration. The vectors used in the methods of gene delivery can vary and can be for example viral vectors such as adenoviruses, adeno-associated viruses (AAV), alphaviruses, herpes viruses, retroviruses/lentiviruses, vaccinia virus, nanostructured non-viral vectors, naked DNA, oligonucleotides, liposomes or particles made with lipoplexes and polyplexes, or a combination thereof (see for example Yazdani et al. Int J Med Rev 2018 September; 5(3):106-117). The developments in the field has led to approval of a gene therapy treatment with AAV for lipoprotein lipase deficiency (alipogene tiparvovec or Glybera®) and is expected to lead to the approval of onasemnogene abeparvovec (Zolgensma), i.e. non replicating recombinant self-complementary (sc) adeno-associated virus serotype 9 (AAV9) vector containing the cDNA of the survival of motor neuron 2 (SMN2) gene under the control of the hybrid cytomegalovirus (CMV) enhancer/chicken beta-actin promoter (CBA), which is to be used for the treatment of as a gene therapy substance in spinal muscular atrophy (WHO Drug Information, Vol. 31, No. 2, 2017).

With the increased use of the aforementioned technologies the abundant availability of vaccines and gene therapy vectors is critical. It is paramount to be able to produce sufficient volumes and quantities of the vaccines and vectors—at the required quality. The same applies to production of therapeutic recombinant proteins. Just as with other parenteral therapeutic products, the purity of a clinical-grade product is crucial. With the complexity of the products their impurity profile also becomes more complex and the purification of a large-scale production more demanding. Removal of impurities that arise during production—without markedly decreasing particle yields—is pivotal to ensure the efficacy and safety of the products. This includes removal of nucleic acid impurities. The preparation of gene therapy pharmaceutical compositions as well as vaccines and proteins includes the use of recombinant techniques being dependent on human, viral, bacterial or artificial genetic material, such as plasmids, or utilizes host cells for growth, or at least is exposed to external bacterial or viral contaminants. As such, nucleic acid impurities of a human or non-human origin in a pharmaceutical composition pose a risk of genotoxicity and immunotoxicity, particularly as such products are administered parenterally. While residual host cell DNA/RNA and residual plasmid DNA pose a risk of genotoxicity, residual nucleic acids of helper viruses can lead to immunotoxicity, genotoxicity and infectious risk (see Wright in Biomedicines 2014, 2, 80-97). DNA contaminants in pharmaceutical compositions may encode oncogenic proteins or regulatory RNAs. The Wright reference also gives an example of the required purification power and specificity that is required to purify AAV vectors generated in cell culture using helper virus-free transient transfection of Human Embryonic Kidney 293 (HEK293) cells. According to that example approximately 10 mg of an AAV vector drug substance containing ˜6.5 mg of AAV capsid protein and ˜3.5 mg of vector DNA must be purified from a cell culture milieu containing ˜4 g of non-vector protein (3 g of HEK293 cellular protein from ˜1010 cells and ˜1 g of fetal bovine serum protein) and ˜350 mg of non-vector nucleic acids (320 mg of HEK293 cellular nucleic acids and 30 mg of production plasmid DNA). It is clear that nucleic acid impurities represent a substantial burden in preparation of the aforementioned products. It is evident that nucleic acid impurities in amount of about 100-times the amount of the product's nucleic acids need to be removed.

Currently, nucleases are often employed to remove nucleic acid impurities during the preparation of vectors transporting nucleic acids. Most often employed are non-specific endonucleases that attack and degrade DNA and RNA (single stranded, double stranded, linear and circular) and remain effective over a wide range of operating conditions. One such endonuclease is Benzonase®, which digests nucleic acids to 5′ monophosphate terminated oligonucleotides of 2-5 bases in length (Janning et al. Rapid Commun. Mass Spectrom. 1994, 8, 1035-1040).

However, due to their preferences for a specific sequence or occasional conformation of nucleic acids nucleases, and limitations of other purification steps such as chromatography, existing purification methods fail to completely digest or remove nucleic acid impurities and leave a significant portion of residual nucleic acids in the final product. The presence of DNA contaminants in pharmaceutical compositions thus remains a major safety concern. Furthermore, due to nucleases' splitting action the nucleic acids impurities are cut and thus cannot be further used as an analyte for an intraprocess control.

### SUMMARY OF THE DISCLOSURE

Improved methods for purifying recombinant protein, vaccine and gene therapy preparations, such as vectors in a suspension, are needed to increase the quality and safety of the pharmaceutical compositions used in the treatment or prevention. The present invention provides new means to purify a liquid composition comprising genetically engineered particles from nucleic acid impurities. It has been surprisingly found that a Dps protein can be added during the purification process to a liquid composition comprising genetically engineered particles to substantially remove nucleic acid impurities.

Therefore, the first aspect of the present disclosure is a method for purifying a liquid composition comprising genetically engineered particles from nucleic acid impurities comprising the steps of (i) adding a Dps protein to the suspension comprising genetically engineered particles, (ii) precipitating a complex comprising the nucleic acid impurities and the Dps protein and (iii) removing the precipitated complex comprising the nucleic acid impurities and Dps protein from the liquid composition comprising genetically engineered particles.

Dps proteins are an inert reagent that does not limit the use of other purification methods and can be easily added to a protocol for purifying particles such as vaccine particles, cell vectors or virus vectors. The Dps protein's ability to bind and precipitate free-floating DNA can be used to supplement or replace the use of a nuclease. An important advantage of the present invention is that a Dps protein can also remove nuclease resistant nucleic acid impurities. Therefore, applying both, a Dps protein and a nuclease in separate purification steps has an added effect. An additional advantage of using a Dps protein to remove nucleic acid impurities from a liquid composition comprising genetically engineered particles is that the Dps protein binds to nucleic acids without any sequence or length specificity and as such offers a robust tool to be used to purify nucleic acid impurities from any expression systems. Given that Dps proteins are prokaryotic proteins they can be produced relatively cheaply in large quantities. As such, they offer a purification solution that is perfectly scalable and can be applied to larger volumes. A further advantage is that a Dps protein instantly forms a complex with a soluble nucleic acid impurities, which precipitates. The rapidness of the precipitation reaction shortens the overall purification time, which is of significant relevance when purifying commercial quantities of a feedstream comprising a recombinant protein, a vaccine or a gene therapy vector. The fact that a Dps protein physically scavenges for residual nucleic acids in a solution and forms with them a precipitating complex offers an elegant solution to remove the formed complex physically from the feedstream; depending on the product preparation for example by filtration or centrifugation. Therefore, the resulting preparation can be purified and washed by much smaller quantity of buffers, which in turn reduces purification time and cost of preparing a pharmaceutical composition comprising a recombinant protein, a vaccine or a gene therapy vector. Compared to the precipitation of nucleic acids with chemical reagents, such as salts or phenols, a Dps protein provides means to form a precipitating complex with nucleic acid (biocrystal) without affecting or disrupting other particles or molecules in a suspension or solution. The use of a Dps protein is also compatible with other purification methods. Conversely, the use of e.g. salts, such as salts of weak acids with strong bases at high concentration (e.g. above 10 mM, or 50 mM, or above 100 mM) to cause precipitation can disrupt genetically engineered particles in the mixture. For example, higher concentrations can cause cell lysis or disrupt virus particles or lead to protein denaturation.

Further aspect of the present invention is a liquid composition comprising genetically engineered particles that is obtained by the aforementioned method.

Another aspect of the present invention is a liquid composition comprising genetically engineered particles or a pharmaceutical composition comprising genetically engineered particles, wherein the liquid composition comprising genetically engineered particles or the pharmaceutical composition comprising genetically engineered particles has been treated with a Dps protein.

Another aspect of the present invention is a pharmaceutical composition comprising genetically engineered particles

A yet another aspect of the present disclosure is a mixture comprising a composition comprising genetically engineered particles and a Dps protein.

A further aspect of the present invention is a mixture comprising a pharmaceutical composition comprising genetically engineered particles and a Dps protein.

Another aspect of the present invention is use of a Dps protein as a purification agent.

Applying the same methods comprising a Dps protein, together with all the advantages described above, allows in turn also to assay the removed nucleic acids. A Dps protein binds very specifically to nucleic acids of all sizes in a sequence independent manner. Therefore, the method of capturing residual amounts of nucleic acid impurities can be turned into a quality control method. Namely, the quality control method can be used to quantify or identify residual amounts of nucleic acid impurities in a sample to evaluate efficiency of a purification process of nucleic acids and to test, e.g. in the bulk preparation or a pharmaceutical composition, if criteria for patient safety in case of therapeutic products have been met. Again, this offers an advantage over using a nuclease, since a Dps protein leaves the removed nucleic acids intact and does not digest them like a nuclease does. Furthermore, the scalability of the method allows to capture nucleic acid impurities from a larger volume and thus significantly increases the detection (e.g. the limit of detection, sensitivity of the analytical protocol) of impurities.

Therefore, another aspect of the present invention is a method to assay residual nucleic acid impurities in a liquid composition comprising genetically engineered particles or a pharmaceutical composition comprising genetically engineered particles, the method comprising the steps of:

(i) adding a Dps protein to the composition or the pharmaceutical composition comprising genetically engineered particles,
(ii) precipitating the residual nucleic acid impurities with the Dps protein,
iii) collecting the precipitated nucleic acid impurities, and
(iv) assaying the residual nucleic acid impurities.
The aspects, advantageous features and preferred embodiments of the present invention summarized in the following items, respectively alone or in combination, further contribute to solving the object of the invention:
1. A method for purifying a liquid composition comprising genetically engineered particles from nucleic acid impurities comprising the steps of (i) adding a Dps protein or a Dps-like protein to the liquid composition comprising genetically engineered particles, (ii) precipitating a complex comprising the nucleic acid impurities and the Dps protein or the Dps-like protein and (iii) removing the precipitated complex comprising the nucleic acid impurities and Dps protein from the composition.
2. The method for purifying a liquid composition comprising genetically engineered particles from nucleic acid impurities according to item 1, wherein the method further comprises a step of skimming, separation funnel separation, filtration, ultrafiltration, mixer-settler separation, centrifugation, ultracentrifugation, chromatography, use of a magnetic particle technology, utilization of magnetic properties of the Dps protein or the Dps-like protein, sterilization and/or treatment of the liquid composition comprising genetically engineered particles with a nuclease, or a combination thereof.
3. The method for purifying a liquid composition comprising genetically engineered particles from nucleic acid impurities according to item 1 or item 2, wherein the steps as defined in item 1 are repeated more than once, optionally at different stages of the method for purifying the liquid composition comprising genetically engineered particles.
4. The method for purifying a liquid composition comprising genetically engineered particles from nucleic acid impurities according to any one of items 1 to 3, where the method comprises adding a nuclease to the liquid composition comprising genetically engineered particles.
5. The method for purifying a liquid composition comprising genetically engineered particles from nucleic acid impurities according to item 4, where the nuclease is an endonuclease.
6. The method for purifying a liquid composition comprising genetically engineered particles from nucleic acid impurities according to item 4 or item 5, where the nuclease is a non-specific endonuclease.
7. The method for purifying a liquid composition comprising genetically engineered particles from nucleic acid impurities according to any one of items 4 to 6, wherein the step of adding the Dps protein or the Dps-like protein precedes the step of adding the nuclease to the liquid composition comprising genetically engineered particles.
8. The method for purifying a liquid composition comprising genetically engineered particles from nucleic acid impurities according to any one of items 4 to 6, wherein the Dps protein or the Dps-like protein is added to the liquid composition comprising genetically engineered particles after the nuclease has been added.
9. The method for purifying a liquid composition comprising genetically engineered particles from nucleic acid impurities according to any one of items 1 to 8, wherein the precipitated nucleic acid and Dps protein are removed from the liquid composition comprising genetically engineered particles by filtration.
10. The method for purifying a liquid composition comprising genetically engineered particles from nucleic acid impurities according to any one of items 1 to 9, wherein the precipitated nucleic acid and Dps protein are removed from the liquid composition comprising genetically engineered particles by centrifugation.
11. The method for purifying a liquid composition comprising genetically engineered particles from nucleic acid impurities according to any one of items 2 to 10, wherein the chromatography comprises affinity column chromatography, hydroxyapatite chromatography, and/or ion-exchange chromatography.
12. The method for purifying a liquid composition comprising genetically engineered particles from nucleic acid impurities according to any one of items 2 to 11, wherein the chromatography comprises ion-exchange chromatography.
13. The method for purifying a liquid composition comprising genetically engineered particles from nucleic acid impurities according to any one of items 2 to 12, wherein the filtration comprises normal flow filtration, dead-end filtration, tangential flow filtration, diafiltration, ultrafiltration, or a combination thereof.
14. The method for purifying a liquid composition comprising genetically engineered particles from nucleic acid impurities according to any one of items 1 to 13, wherein the nucleic acid impurity is virus or mammalian DNA, or a fragment thereof, preferably is virus DNA.
15. The method for purifying a liquid composition comprising genetically engineered particles from nucleic acid impurities according to any one of items 1 to 14, wherein the nucleic acid impurity is resistant to a nuclease.
16. The method for purifying a liquid composition comprising genetically engineered particles from nucleic acid impurities according to any one of items 1 to 15, wherein the concentration of Mg2+ ions in the liquid composition comprising genetically engineered particles during the step of precipitating the complex comprising the nucleic acid impurities and the Dps protein or the Dps-like protein is not more than 7.5 mM, preferably not more than 3.75 mM.
17. The method for purifying a liquid composition comprising genetically engineered particles from nucleic acid impurities according to item 16, wherein the concentration of Mg2+ is not more than 2 mM, preferably is between 1 and 2 mM.
18. The method for purifying a liquid composition comprising genetically engineered particles from nucleic acid impurities according to item 16 or item 17, wherein the concentration of Mg2+ ions at the time when nuclease is added is different from said concentration during the step of precipitating the complex comprising the nucleic acid impurities and the Dps protein or the Dps-like protein.
19. The method for purifying a liquid composition comprising genetically engineered particles from nucleic acid impurities according to any one of preceding items, wherein the volume of the liquid composition comprising genetically engineered particles is up to about 1000 L.
20. The method for purifying a liquid composition comprising genetically engineered particles from nucleic acid impurities according to any one of preceding items, wherein the volume of the liquid composition comprising genetically engineered particles to which the Dps protein or the Dps-like protein is added is up to 1000 L.
21. The method for purifying a liquid composition comprising genetically engineered particles from nucleic acid impurities according to any one of the preceding items, wherein the volume of the liquid composition comprising genetically engineered particles is from about 5 mL and up to about 100 L.
22. The method for purifying a liquid composition comprising genetically engineered particles from nucleic acid impurities according to any one of preceding items, wherein the volume of the liquid composition comprising genetically engineered particles is from about 500 mL and up to about 25 L, preferably from about 500 mL to about 10 L.
23. The method for purifying a liquid composition comprising genetically engineered particles from nucleic acid impurities according to any one of the preceding items, wherein the nucleic acids impurities from the removed precipitated complex are further assayed.
24. The method for purifying a liquid composition comprising genetically engineered particles from nucleic acid impurities according to item 23, wherein the nucleic acids impurities are quantified.
25. The method for purifying a liquid composition comprising genetically engineered particles from nucleic acid impurities according to item 23 or item 24, wherein a source of the nucleic acids impurities is further identified.
26. The method for purifying a liquid composition comprising genetically engineered particles from nucleic acid impurities according to any one of the preceding items, further preparing a pharmaceutical composition comprising the genetically engineered particles.
27. The method for purifying a liquid composition comprising genetically engineered particles from nucleic acid impurities according to item 26, wherein the pharmaceutical composition is prepared by partitioning, mixing the liquid composition comprising genetically engineered particles with a pharmaceutically acceptable excipient, labelling, or a combination thereof.
28. A liquid composition comprising genetically engineered particles obtained by a method according to any one of items 1 to 25.
29. A pharmaceutical composition comprising genetically engineered particles obtained by a method according to item 26 or item 27.
30. A mixture comprising a liquid composition comprising genetically engineered particles and a Dps protein or a Dps-like protein.
31. A mixture comprising a pharmaceutical composition comprising genetically engineered particles and a Dps protein or a Dps-like protein.
32. A liquid composition comprising genetically engineered particles or a pharmaceutical composition comprising genetically engineered particles, wherein the liquid composition comprising genetically engineered particles or the pharmaceutical composition comprising genetically engineered particles has been treated with a Dps protein or a Dps-like protein.
33. The liquid composition comprising genetically engineered particles or the pharmaceutical composition comprising genetically engineered particles according to item 32, wherein the liquid composition comprising genetically engineered particles or the pharmaceutical composition comprising genetically engineered particles has been further treated with a nuclease.
34. Use of a Dps protein or a Dps-like protein as a purification reagent.
35. The use of a Dps protein or a Dps-like protein according to item 34 in a liquid composition comprising genetically engineered particles.
36. The use according to item 34 or item 35 comprising adding the Dps protein or the Dps-like protein to the composition comprising a genetically engineered particles to remove nucleic acid impurities.
37. The use of a Dps protein or a Dps-like protein according to any one of items 34 to 36 in combination with a nuclease.
38. The use according to item 37, wherein the Dps protein or the Dps-like protein and the nuclease are used sequentially.
39. The use according to item 37 or item 38, wherein the nuclease is endonuclease.
40. The use according to any one of items 37 to 39, wherein the nuclease is non-specific endonuclease.
41. The use of a Dps protein or a Dps-like protein according to any one of items 34 to 40 comprising the steps of (i) mixing a Dps protein or a Dps-like protein and a liquid composition comprising genetically engineered particles, (ii) precipitating a complex comprising nucleic acid impurities and the Dps protein or the Dps-like protein and (iii) removing the precipitated complex comprising the nucleic acid impurities and Dps protein from the liquid composition comprising genetically engineered particles.
42. The use of a Dps protein or a Dps-like protein according to any one of items 36 to 41, wherein the nucleic acid impurities are resistant to a nuclease.
43. A method to assay nucleic acid impurities in a liquid composition comprising genetically engineered particles or a pharmaceutical composition comprising genetically engineered particles, the method comprising the steps of:
(i) adding a Dps protein or a Dps-like protein to the composition or the pharmaceutical composition comprising genetically engineered particles,
(ii) precipitating the nucleic acid impurities with the Dps protein or the Dps-like protein,
iii) collecting the precipitated nucleic acid impurities, and
(iv) assaying the nucleic acid impurities.
44. The method to assay nucleic acid impurities in a liquid composition comprising genetically engineered particles or a pharmaceutical composition comprising genetically engineered particles according to item 43, wherein the nucleic acid impurities are further released from the precipitate with the Dps protein or the Dps-like protein.
45. The method to assay nucleic acid impurities in a liquid composition comprising genetically engineered particles or a pharmaceutical composition comprising genetically engineered particles according to item 44, wherein the nucleic acid impurities are released from the precipitate with the Dps protein or the Dps-like protein by addition of proteinase or divalent cations to the precipitate or by precipitation of the nucleic acid impurities.
46. The method to assay nucleic acid impurities in a liquid composition comprising genetically engineered particles or a pharmaceutical composition comprising genetically engineered particles according to item 44, wherein the nucleic acid impurities are released from the precipitate with the Dps protein or the Dps-like protein by addition of proteinase.
47. The method to assay nucleic acid impurities in a liquid composition comprising genetically engineered particles or a pharmaceutical composition comprising genetically engineered particles according to item 46, wherein the proteinase is proteinase K or proteinase A.
48. The method to assay nucleic acid impurities in a liquid composition comprising genetically engineered particles or a pharmaceutical composition comprising genetically engineered particles according to item 44, wherein the nucleic acid impurities are released from the precipitate with the Dps protein or the Dps-like protein by precipitation.
49. The method to assay nucleic acid impurities in a liquid composition comprising genetically engineered particles or a pharmaceutical composition comprising genetically engineered particles according to item 48, the precipitation is ethanol or phenol precipitation, preferably is phenol precipitation.
50. The method to assay nucleic acid impurities in a liquid composition comprising genetically engineered particles or a pharmaceutical composition comprising genetically engineered particles according to item 44, wherein the nucleic acid impurities released from the precipitate with the Dps protein or the Dps-like protein by adjusting the concentration of divalent cations.
51. The method to assay nucleic acid impurities in a liquid composition comprising genetically engineered particles or a pharmaceutical composition comprising genetically engineered particles according to any one of items 43 to 50, where the nucleic acid impurities are quantified.
52. The method to assay nucleic acid impurities in a liquid composition comprising genetically engineered particles or a pharmaceutical composition comprising genetically engineered particles according to any one of items 43 to 51, where a source of the nucleic acid impurities is identified.
53. The method to assay nucleic acid impurities in a liquid composition comprising genetically engineered particles or a pharmaceutical composition comprising genetically engineered particles according to any one of items 43 to 52, where the nucleic acid impurities are assayed by a PCR-based technique.
54. The method to assay nucleic acid impurities in a liquid composition comprising genetically engineered particles or a pharmaceutical composition comprising genetically engineered particles according to any one of items 43 to 53, wherein the nucleic acid impurities are assayed by sequencing.
55. The method for purifying a liquid composition comprising genetically engineered particles from nucleic acid impurities according to any one of items 1 to 27, the liquid composition comprising genetically engineered particles according to any one of items 28, 32 or 33, the pharmaceutical composition comprising genetically engineered particles according to any one of items 29, 32 or 33, the mixture composition comprising genetically engineered particles and a Dps protein or a Dps-like protein according to item 30 or item 31, the use of a Dps protein or a Dps-like protein according to any one of items 34 to 42, or the method to assay nucleic acid impurities in a liquid composition comprising genetically engineered particles or a pharmaceutical composition comprising genetically engineered particles according to any one of items 43 to 55, wherein the genetically engineered particles are AAV.
56. The method for purifying a liquid composition comprising genetically engineered particles from nucleic acid impurities according to any one of items 1 to 27, the liquid composition comprising genetically engineered particles according to any one of items 28, 32 or 33, the pharmaceutical composition comprising genetically engineered particles according to any one of items 29, 32 or 33, the mixture composition comprising genetically engineered particles and a Dps protein or a Dps-like protein according to item 30 or item 31, the use of a Dps protein or a Dps-like protein according to any one of items 34 to 42, or the method to assay nucleic acid impurities in a liquid composition comprising genetically engineered particles or a pharmaceutical composition comprising genetically engineered particles according to any one of items 43 to 55, wherein the genetically engineered particles are a prokaryotic cell, an eukaryotic cell, a virus, a bacteriophage, a nanostructured particle, an extracellular vesicle or a liposome, or a combination thereof.
57. The method for purifying a liquid composition comprising genetically engineered particles from nucleic acid impurities according to any one of items 1 to 27 or 56, the liquid composition comprising genetically engineered particles according to any one of items 28, 32, 33 or 56, the pharmaceutical composition comprising genetically engineered particles according to any one of items 29, 32, 33 or 56, the mixture composition comprising genetically engineered particles and a Dps protein or a Dps-like protein according to any one of items 30, 31 or 56, the use of a Dps protein or a Dps-like protein according to any one of items 34 to 42 or 56, or the method to assay nucleic acid impurities in a liquid composition comprising genetically engineered particles or a pharmaceutical composition comprising genetically engineered particles according to any one of items 43 to 56, wherein the nucleic acid impurity is DNA, optionally viral DNA.
58. The method for purifying a liquid composition comprising genetically engineered particles from nucleic acid impurities according to any one of items 1 to 27, 56 or 57, the liquid composition comprising genetically engineered particles according to any one of items 28, 32, 56 or 57, the pharmaceutical composition comprising genetically engineered particles according to any one of items 29, 32, 33, 56 or 57, the mixture composition comprising genetically engineered particles and a Dps protein or a Dps-like protein according to any one of items 30, 31, 55 or 56, the use of a Dps protein or a Dps-like protein according to any one of items 34 to 42, 56 or 57, or the method to assay nucleic acid impurities in a liquid composition comprising genetically engineered particles or a pharmaceutical composition comprising genetically engineered particles according to any one of items 43 to 57, wherein the nucleic acid impurity is genomic or plasmid DNA, or a fragment thereof.
59. The method for purifying a liquid composition comprising genetically engineered particles from nucleic acid impurities according to any one of items 1 to 27, 56 or 57, the liquid composition comprising genetically engineered particles according to any one of items 28, 32, 56 or 57, the pharmaceutical composition comprising genetically engineered particles according to any one of items 29, 32, 33, 56 or 57, the mixture composition comprising genetically engineered particles and a Dps protein or a Dps-like protein according to any one of items 30, 31, 55 or 56, the use of a Dps protein or a Dps-like protein according to any one of items 35 to 42, 56 or 57, or the method to assay nucleic acid impurities in a liquid composition comprising genetically engineered particles or a pharmaceutical composition comprising genetically engineered particles according to any one of items 43 to 57, wherein the liquid composition is a solution.
60. The method for purifying a liquid composition comprising genetically engineered particles from nucleic acid impurities according to any one of items 1 to 27, 56 or 57, the liquid composition comprising genetically engineered particles according to any one of items 28, 32, 56 or 57, the pharmaceutical composition comprising genetically engineered particles according to any one of items 29, 32, 33, 56 or 57, the mixture composition comprising genetically engineered particles and a Dps protein or a Dps-like protein according to any one of items 30, 31, 55 or 56, the use of a Dps protein or a Dps-like protein according to any one of items 35 to 42, 56 or 57, or the method to assay nucleic acid impurities in a liquid composition comprising genetically engineered particles or a pharmaceutical composition comprising genetically engineered particles according to any one of items 43 to 57, wherein the liquid composition is a suspension.
61. The method for purifying a liquid composition comprising genetically engineered particles from nucleic acid impurities according to any one of items 1 to 27, 56 or 57, the liquid composition comprising genetically engineered particles according to any one of items 28, 32, 56 or 57, the pharmaceutical composition comprising genetically engineered particles according to any one of items 29, 32, 33, 56 or 57, the mixture composition comprising genetically engineered particles and a Dps protein or a Dps-like protein according to any one of items 30, 31, 55 or 56, the use of a Dps protein or a Dps-like protein according to any one of items 35 to 42, 56 or 57, or the method to assay nucleic acid impurities in a liquid composition comprising genetically engineered particles or a pharmaceutical composition comprising genetically engineered particles according to any one of items 43 to 57, wherein the genetically engineered particle are a recombinant protein.
62. The method for purifying a liquid composition comprising genetically engineered particles from nucleic acid impurities , the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items, wherein the genetically engineered particle is therapeutically effective.
63. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items 1 to 60 or 62, wherein the genetically engineered particle is a virus.
64. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items 1 to 60 or 62, wherein the genetically engineered particle is AAV.
65. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items 1 to 60 or 62, wherein the genetically engineered particle is AAV of serotype 2, 8 or 9.
66. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items 1 to 60 or 62, wherein genetically engineered particle is a retrovirus or a lentivirus, preferably is a lentivirus.
67. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items 1 to 60 or 62, wherein genetically engineered particle is a cell, preferably a mammalian cell.
68. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items 1 to 60, 62 or 67, wherein the genetically engineered particle is a cell expressing chimeric antigen receptor (CAR).
69. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items, wherein the Dps proteins is added at the complex comprising Dps protein andnucleic acid impurity precipitated at a temperature between about 25° C. and 2° C., preferably between about 20° C. and 3° C., preferably between about 10° C. and 4° C., more preferably at about 4° C.
70. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items, wherein the Dps protein is added (i.e. not the Dps-like protein; i.e. the Dps protein excludes the Dps-like protein).
71. The method for purifying a liquid composition comprising genetically engineered particles from nucleic acid impurities, the liquid composition comprising genetically engineered particles, the pharmaceutical composition comprising genetically engineered particles, the mixture composition comprising genetically engineered particles and a Dps protein or a Dps-like protein, the use of a Dps protein or a Dps-like protein, or the method to assay nucleic acid impurities in a liquid composition comprising genetically engineered particles or a pharmaceutical composition comprising genetically engineered particles according to any one of previous items, wherein the Dps protein has a sequence identity of 30% or more with SEQ ID NO: 1, preferably 40% or more, 50% or more, 60% or more, 70% or more, 80% or more, 90% or more, 95% or more, 98% or more with the SEQ ID NO: 1.

### BRIEF DESCRIPTION OF THE DRAWINGS

FIG. 1: A Dps proteins (10 μL) can precipitate various amounts of DNA from 1 mL solution. DNA yields before (empty bars) and after (black bars) separation are comparable, suggesting complete removal of DNA from a solution. Error bars represent 5% coefficient of variation (CV) of the fluorescence measurement.

FIG. 2: Size distribution of spike-in DNA before and after extraction with Dps.

Fragment length distributions were assessed for DNA standard (50 bp DNA ladder) suspended in nuclease-free water (a; provided by DNA ladder manufacturer); and subsequently extracted with Dps (b; visualized with gel electrophoresis).

FIG. 3: Efficient removal of genomic DNA—evidence that a Dps protein binds to genomic DNA Genomic DNA (gDNA) is efficiently removed from a solution using a Dps protein. Over 40-fold difference in total gDNA amount in supernatant is observed between sample A (without the use of a Dps protein) and sample B (after the removal of DNA with Dps), confirming that a precipitating Dps-DNA complex was pelleted during centrifugation.

FIG. 4: Accumulation of nucleic acids from a larger volume

High yield of pure DNA obtained from a larger volume by purifying DNA with both Dps protein and with the QIAamp MinElute ccfDNA Mini kit (QIAGEN) (upper curve), compared to the lower amount of DNA obtained by purifying it solely with the QIAamp MinElute ccfDNA Mini kit (QIAGEN) (bottom curve). Adding a process step of purifying a sample from nucleic acids by using Dps protein to a purification method known in the art allows to collect nucleic acids from volumes larger than those used in the respective purification methods known in the art. In addition, the obtained nucleic acids are of better quality.

### DETAILED DESCRIPTION OF THE INVENTION

The disclosure can be conveniently put into practice by adding a Dps protein to a liquid composition comprising genetically engineered particles comprising free-floating nucleic acid impurities to form a protein-nucleic acid complex. The Dps protein forms a complex with the free-floating nucleic acid impurities. The thus formed complex precipitates and as such can be easily removed from the rest of the suspension comprising genetically engineered particles. Once added to the sample, the Dps protein forms a complex with naked nucleotides comprised in the sample and precipitates them. The binding of the protein to the polynucleotides in the sample and thus forming of the complex protein-nucleid acid can be improved by incubating the sample at lower temperatures. For example, the incubation of the sample below the room temperature, preferably at 4° C. improves the purifying capacity of the Dps protein. The formation of the precipitating complex can dependent on the pH of the suspension comprising genetically engineered particles and concentration of divalent-metal ions. The Dps protein forms the complex with nucleic acids in a wide pH range. Nucleic acids get precipitated at least at pH less than 7, but also higher pH can work; for example between pH 3 and pH 8. It can be also observed in literature that a Dps protein can bind to DNA for example at low pH of 2.2 or pH 3.6. The effects of Mg2+ ions and pH on Dps binding efficacy were tested in Ceci et al. Nucleic Acids Research, Volume 32, Issue 19, 2004, 5935-5944. The concentration of ions can be reduced by adding a chelating agent such as EDTA, or desalinating the suspension comprising genetically engineered particles by diafiltration or dialysis. The ion concentration can also be increased by adding bases, acids or salts thereof to the suspension comprising genetically engineered particles, e.g. to improve efficiency of a nuclease, such as endonuclease, or to attain specific loading or eluting conditions for a later chromatography. Most enzymes that act at phosphodiester bonds in DNA, including the vast majority of restriction endonucleases identified to date (as listed in REBASE database; Robert et al, Nucleic Acids Research, 2007, 35, D269-D270) require divalent metal ions as catalytic cofactors, usually Mg2+ as reviewed in Bellamy et al (Nucleic Acids Research, 2009, 37, 16, 5443-5453) and Yang et al (Molecular Cell, 2006, 22, 5-13). One such nuclease is the Benzonase®, a non-specific endonuclease, which can be applied early in the process. The Benzonase requires Mg2+ ions in concentration between 1-2 mM and is thus fully compatible with the conditions at which a Dps protein can work. The same applies to the preferred pH window.

Generally, the concentration of divalent metal ions needed for optimal binding of a respective Dps protein can be determined by preparing series of standard water solutions comprising nucleic acids, such as a standard ladder of known size and concentration, and increased concentrations of the metal ions. The result of such optimization experiment can also help to define the best stage to add the Dps protein to the suspension comprising genetically engineered particles—so that it complements the rest of the purification steps. Generally, once the complex of the Dps protein and nucleic acid impurity from a suspension comprising genetically engineered particles precipitates, the precipitated complex can be separated from the sample by any known method to separate the precipitate. While the complex between a Dps protein and a nucleic acid in a solution (or suspension) forms instantly, the resulting mixture can be incubated. The incubation time can be for example between 0 minutes to overnight (e.g. 16 hours). The incubation time can for example be 30 minutes, 15 minutes, 10 minutes or 5 minutes. In one embodiment, the incubation time is 10 minutes. In another embodiment, the incubation is 5 minutes. The incubation can include mixing, which can be constant or intermittent, and be combined with any degree of cooling at temperatures above the freezing point and below 25° C., for example at about 4° C. In one embodiment, the complex formation in a solution (suspension) is conducted at temperatures below 10° C., preferably at about 4° C. The same temperature of the mixture can be retained during the step of complex removal. As an example, the Dps protein-nucleic acid complex can be separated by chromatography (e.g. using Sephadex G-15 column), centrifugation, sedimentation, filtration of the sample, use of magnetic particle technologies, or by utilizing magnetic properties of the Dps protein. Another option to separate assembled complex from the sample when a Dps protein is used is by utilizing magnetic properties of the Dps protein. Dps proteins have the ability to oxidize Fe2+ to paramagnetic Fe3+, which is deposited inside a protein cavity. This results in the formation of an iron core with magnetic properties, which can be exploited for separation of Dps protein-nucleic acid complexes. Preferably, the complex is separated by centrifugation. Centrifugation is deemed as one of the basic laboratory techniques.

The term “a liquid composition” as used herein refers to liquid mixture, such as a solution or a suspension. It will be apparent to a skilled person that while the composition at a certain stage in preparation or purification, or the resulting composition (or even a pharmaceutical composition) may be solid, liquid or semi-solid, the methods of the present disclosure can be implemented when a Dps protein can act in a solution or a suspension. Therefore, it is best if the composition comprising genetically engineered particles is liquid (e.g. a solution or a suspension) at least during the step when the complex between a Dps protein and nucleic acid impurities is formed and precipitated (optionally only intermittently or with interruptions). Depending on the type or nature of a genetically engineered particle the liquid composition will be either a solution or a suspension, or will change from one to the other during the execution of a method of the present disclosure. For example, a recombinant protein in a buffer can form a solution. However, depending on conditions used, the same proteins may also form a suspension. Cells and viruses normally form a suspension. The type of the mixture, i.e. whether the liquid composition will form a solution or a suspension, will depend also on components other than generically engineered particles in the compositions, such as cell debris, other solutes, etc. The term “genetically engineered particle”, as used herein relates to a recombinant protein or a particle comprising a genetically engineered nucleic acid or a recombinant protein. The term can mean a particle comprising a genetically engineered nucleic acid or a recombinant protein, preferably the particle comprises genetically engineered nucleic acid. In one embodiment, a generically engineered particle is a recombinant protein. In another embodiment, the particle comprising a genetically engineered nucleic acid can be for example a prokaryotic cell, eukaryotic cell, a virus, a bacteriophage, a nanostructured particle, an extracellular vesicle or a liposome, or a combination thereof, preferably it is a prokaryotic cell, eukaryotic cell, or a virus, more preferably it is a virus. The eukaryotic cell can be for example an animal, insect, or a plant cell. The term liquid composition comprising genetically engineered particles can for example relate to a suspension at any step of a purification process after the first harvest of a cell or a virus or preparation of a nanostructured particle or a liposome. The cells are for example bacterial or mammalian cells intended to be used in therapy. Viruses that can be purified with the method of the present disclosure include but are not limited to negative strand RNA viruses, including but not limited to influenza, RSV, parainfluenza viruses 1, 2 and 3, and human metapneumovirus, as well as other viruses, including DNA viruses, retroviruses, positive strand RNA viruses, negative strand RNA viruses, double-stranded RNA viruses, including, but not limited to, papovavirus, vesicular stomatitis virus, vaccinia virus, Coxsackie virus, reovirus, parvovirus, adenovirus, adenovirus-associated virus (AAV), poliomyeltitis virus, measles virus, rabies virus, and herpes virus. The genetically engineered particles can comprise live recombinant vaccines, i.e. a live viral or bacterial vector engineered to express an exogenous antigen in the cytoplasm of a target cell. Vectors used to prepare recombinant vaccines can be for example Bacillus Calmette-Guérin (NIH; Japan), Salmonella (IAVI; University of Maryland), Other bacterial vectors: Lactobacillus, Streptococcus, Listeria, Poxvirus vectors (MVA, NYVAC, fowlpox and canarypox [ALVAC] viruses), Human ΔE1A replication-defective adenoviruses A, Replication-competent human, Nonreplicating chimpanzee adenoviruses, Picornavirus vectors (poliovirus, rhinovirus), Venezuelan equine encephalitis virus (Alphavax), Adeno-associated virus (IAVI; targeted genetics), Sendai virus (NIH; Japan), Vesicular stomatitis virus (Yale University; Wyeth), Newcastle disease virus (Mount Sinai, New-York; Kyoto University, Japan) Measles virus (Pasteur Institute; GlaxoSmithKline), Rhesus cytomegalovirus (Oregon Health and Science University) (see Human immunodeficiency virus vaccines in Marc P. Girard, Wayne C. Koff, in Vaccines (Sixth Edition), 2013). The present methods can be applied to the preparation and purification of genetically engineered and thus modified cells, such as lymphocytes, e.g., a T lymphocyte, which may either be obtained from a patient or a donor. The cell can be autologous or allogeneic. The cell may be modified to express an exogenous construct, such as, e.g., a chimeric antigen receptor (CAR) or a T cell receptor (TCR), which is incorporated into the cell's genome. One example are CAR-T cells. The cell may also be a B lymphocyte, natural killer (NK) cell, macrophage, eosinophil, mast cell, dendritic cell or a neutrophil. In another embodiment, the cell is a bacterial cell. The CAR can be an extracellular single chain variable fragment (scFv) with specificity and affinity for a particular tumor antigen, or antigen in a disease other than cancer, linked to an intracellular signaling part comprising at least one costimulatory domain and at least one activating domain. The CAR scFv may target, for example, CD19, which is a transmembrane protein expressed by cells in the B cell lineage, including all normal B cells and B cell malignances, including but not limited to NHL, CLL, and non-T cell ALL. Example CART cell therapies and constructs are described in U.S. Patent Publication Nos. 2013/0287748, 2014/0227237, 2014/0099309, 2014/0050708, or WO/2019/094360, WO/2019/089982, or WO/2019/089798.

Extracellular vesicles that can be purified by the methods disclosed herein and can be used for nucleic acid delivery and be suitable for RNA-based gene therapy (Jiang et al. Gene Therapy, 2017, 24, pages 157-166).

The virus can be harvested for example from a host cells, which can be for example an insect cell, a mammalian cell or a prokaryote cell. The host-cells to produce viruses can be for example CHO, Escherichia coli, Human, Vero, Pichia, NS0, MDCK, HeLa, A549, BHK, Sf9, or HEK293 cell line. The first harvest can include lysis of host-cells, but also encompasses harvesting viruses without host-cell lysis.

In a preferred embodiment, the methods of the present disclosure are applied to purify AAV preparations. Robert et all in Biotechnol. J. 2017, 12, 1600193 reviewed articles like for example Balakrishnan B., Jayandharan G. R., Curr. Gene Ther. 2014, 14, 86-100; Weitzman M. D., Linden R. M., Methods Mol. Biol. 2011, 807, 1-23; Hölscher C. et al. J. Virol. 1995, 69, 6880-6885; Hamilton, et al. J. Virol. 2004, 78, 7874-7882; Dong B., et al Mol. Ther. 2010, 18, 87-92; and Wu Z et al. Mol. Ther. 2010, 18, 80-86 that provide adeno-associated virus biology and characterization. AAV is a non-enveloped icosahedral particle (20-25 nm in diameter) containing a positive or a negative sense single-stranded DNA genome. AAV depend on a helper virus such as adenovirus or herpesvirus to provide essential genes in trans for productive infection. The wild-type AAV contains two genes, the rep and cap genes, that express proteins necessary for viral replication, viral capsid proteins, packaging of the viral genome and viral latency. AAV are produced by providing the viral functions in trans (rep, cap and helper viral genes) except for the two inverted terminal repeats (ITRs, required for viral genome replication). The preparation of AAV is described for example in Strobel et al. Human gene therapy methods, 30, 2019; Chahal et al. Journal of Virological Methods, 196 (2014) 163-173, Shin et al., Methods Mol Biol. 2012; 798: 267-284., or Robert et al. Biotechnol. J. 2017, 12, 1600193. AAV has enough space for insertion of 4.4 to 4.7 kb single-stranded recombinant DNA. In the case of self-complementary AAV (scAAV), where the complementary strand is packaged as double-stranded scAAV in the capsid, the scAAV can carry only half of the genome size of a single stranded AAV, i.e. about 2.4 kb. There are at least 100 different variant (naturally occurring or synthesized) capsid sequences of AAV known. However, the most commonly applied serotypes are the eleven AAV serotypes (AAV1 to AAV11) that were isolated from human, simian, and rhesus and cynomolgus monkeys. The AAV can be of any serotype, for example of a serotype AAV1, AAV2, AAV3B, AAV4, AAV5, AAV6, AAV8, or AAV9. The preferred AAV serotypes are AAV2, AAV8 or AAV9. The serotypes differ in their tropism, or the types of cells they infect best. Depending on the target organ tissue, a certain serotype can be selected. For example, for CNS tissue, AAV1, AAV2, AAV4, AAV5, AAV8 or AAV9; heart tissue: AAV1, AAV8 or AAV9; kidney tissue: AAV2; liver tissue: AAV7, AAV8 or AAV9; lung tissue: AAV4, AAV5, AAV6 or AAV9; pancreas: AAV8; photoreceptor cells: AAV2, AAV5 or AAV8; retinal pigment epithelium: AAV1, AAV2, AAV4, AAV5 or AAV8; skeletal muscle: AAV1, AAV6, AAV7, AAV8 or AAV9. More details can be obtained from Chahal P. S. et al. in Journal of Virological Methods 196 (2014) 163-173.

In another embodiment, the present methods are used to purify a virus vector, which is prepared to transfect a cell in a subject to cause expression of a gene or suppression of expression of a detrimental gene by employing RNA interference or genome editing tools. In one embodiment, the genetically engineered particle takes part in any one of programmable nuclease-based methods (for example, CRISPR-Cas9). For example, the generically engineered particle can edit genetic material in a cell it transfects (as an example, Iyer S. et al in Nature, 2019, 568, 561-565).

In one embodiment, the term excludes genetically engineered particles comprising a Dps protein packed around genetically engineered nucleic acids, or genetically engineered Dps packed with nucleic acids. In another embodiment, in a situation where the genetically engineered particles in a suspension to be purified already comprise a first Dps protein and nucleic acids, then a Dps protein used in the purification or assay methods, or products, according the present invention, is a second Dps protein and different from the first Dps forming the particles to be purified. The second Dps protein should be different from the first Dps to allow selective removal of a complex of the second Dps protein with the nucleic acid impurity after being added to the composition comprising the genetically engineered particles (i.e. the first Dps with nucleic acids).

The terms “peptide”, “polypeptide” and “protein” are used interchangeably, and refer to a compound comprised of amino acid residues covalently linked by peptide bonds. Polypeptides include any peptide or protein comprising two or more amino acids, preferably five or more, more preferably 10 or more, joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” and “protein” include, for example, biologically active fragments, substantially homologous polypeptides, oligopeptides, homodimers, heterodimers, variants of polypeptides, modified polypeptides, derivatives, analogs, fusion proteins, or combinations thereof. In one embodiment, a recombinant protein is an antibody, or a modification or a fragment thereof. The recombinant protein can be for example a Fab fragment, or a bivalent and bispecific antibody or its fragment (Holliger P et al. Proc Natl Acad Sci U S A. 1993 Jul. 15; 90(14): 6444-6448). Proteins can be produced using techniques well known in the art, e.g., recombinant technologies, phage display technologies, synthetic technologies or combinations of such technologies or other technologies readily known in the art (see, for example, Jayasena, S. D., Clin. Chem., 45: 1628-50, 1999 and Fellouse, F. A., et al, J. Mol. Biol., 373(4):924-40, 2007). For example, antibodies can be prepared by the hybridoma method first described by Kohler and Milstein, Nature 256:495, 1975, or may be made by recombinant DNA methods such as described in U.S. Pat. No. 4,816,567.

The term “genetically engineered” or “recombinant” refers to a method of modifying the genome of a cell, or the sequence of a vector, such as a plasmid, including, but not limited to, deleting a coding or non-coding region or a portion thereof or inserting a coding region or a portion thereof; and results of such method, such as constructed nucleic acid sequence, or once this is expressed and translated, a (recombinant) protein. In one embodiment, the result of such methods is a constructed nucleic acid (e.g. a virus comprising such constructed nucleic acid). In one embodiment, it relates to a nucleic acid, a virus particle, or a protein that does not occur in nature.

A “host cell” includes an individual cell or a cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation.

The term “Dps protein” as used herein refers to a DNA-binding protein from starved cells (Dps). The term can include fragments, homologous proteins, homodimers, heterodimers, variants, modified Dps proteins, derivatives, analogs, fusion proteins, or combinations thereof. Dps proteins were first characterized by Almirón M. et al. in A novel DNA-binding protein with regulatory and protective roles in starved Escherichia coli in Genes Dev. (1992) December; 6(12B):2646-54. Dps are proteins found in bacteria (97%) and archaea (3%), but not in animals or humans. Dps proteins are often composed of 12 identical monomers (with molecular mass of about 19 kDa each) assembled into a hollow, tetrahedrally symmetric sphere with a diameter of about 9 nm and an inner cavity of about 4.5 nm in diameter (Grant et al, Nat Struct Biol 1998, 5:294-303). Karas at al (J Bacteriol 197:3206-3215) confirmed that Dps possess the ability of DNA binding and iron oxidation, which are performed completely independently. While the dodecameric nanoparticle structure is well conserved among all Dps, the Dps primary amino acid sequences vary widely among different families of bacteria or archaea, with some sharing less than 30% amino acid sequence identity. For example, Dps-tery as described in Castruita et al, Applied and Environmental Microbiology, April 2006, p. 2918-2924 exhibits 69% primary amino acid sequence identity with Synechococcus DpsA, 32% primary amino acid sequence identity with E. coli Dps, and 30% primary amino acid sequence identity with Listeria innocua Flp. A Dps protein can thus for example exhibit 30% or more % amino acid sequence identity with another Dps protein. Therefore, in one embodiment, a Dps protein can be a protein having 30% or more amino acid sequence identity with SEQ ID NO: 1. A Dps protein can be a protein having 50% or more, 60% or more, or 80% or more or 90% or more amino acid sequence identity with SEQ ID NO: 1. Certain examples of Dps proteins are Dps of E. Coli (Swiss-Prot Accession #P0ABT2), Mycobacterium smegmatis (Swiss-Prot Accession #P00558), 735 Lactobacillus, Bacteroides, extremophilic or hyperthermophilic bacteria or archaea such as Sulfolobus solfataricus or Deinococcus radiodurans. The Dps proteins can be for example those that are found in bacteria strains of Streptococci, Listeria, Helicobacter and Escherichia. Preferably the Dps protein is from Escherichia Coli. In one embodiment, the Dps protein is the protein known under the accession number NP_415333.1. The protein served as an example of a suitable Dps protein that can be applied according to the present disclosure. Based on the present disclosure it would be in a purview of a skilled person to select other suitable Dps proteins, or if needed, to test their suitability for the uses disclosed herein based on their ability to form a precipitating complex with a nucleic acid in water as described below or in the examples. Suitable proteins can be found for example among the proteins of the Dps family in the UniProt Knowledgebase. A Dps protein can also be modified, e.g. by recombinant techniques. The capacity to bind DNA should be retained.

Generally, a Dps protein can form a precipitating complex with nucleic acids in a solution. It is known that Dps binds to polynucleotides without any apparent sequence specificity, forming a highly stable complex and compacting the polynucleotides into a highly ordered crystalline structure (i.e. biocrystals) (Wolf et al., Nature, Vol 400, July 1999). The highly organized co-crystallization process is obtained with supercoiled plasmids, linear double-stranded DNA, single-stranded DNA, as well as single-stranded RNA molecules without a significant difference in the resultant crystalline structure. The crystals result in physical protection of nucleic acids against wide range of stresses, including oxidative stress, high pressure, UV and gamma radiation, pH shock, iron and copper toxicity and high temperatures (i.e. up to 100° C.). In addition to polynucleotide binding, Dps protein can exhibit other properties, such as metal binding and sequestration, ferroxidase activity and ability to affect gene regulation. Dps proteins are mini-ferritins with many structural and functional similarities to maxi-ferritins (Ftn and Bfr proteins) and together they form the ferritin superfamily of iron storage proteins with magnetic properties. The affinity of Dps for polynucleotides is sensitive to buffer conditions. Dps protein binds polynucleotides weaker in the presence of higher salt concentrations. For example, the formation of a complex, i.e. biocrystals, can depend on the concentration of divalent cations such as Mg2+, where a concentration of up to about 3.75 mM Mg2+, preferably up to about 1 mM Mg2+, can support the assembly of protein-polynucleotide complexes, while the concentration of more than about 7.5 mM Mg2+, possibly more than about 3.75 mM Mg2+ can disrupt the formation of the complex (Ceci et al. Nucleic Acids Research, Volume 32, Issue 19, 2004, 5935-5944). In general, for a Dps or a Dps-like protein the ability to precipitate can be confirmed if the protein can precipitate nucleic acids in water at pH 7 when the concentration of nucleic acids in water is at least 12 ng/mL and the concentration of the Dps protein is at least 70 μg/μL, 7 μg/μL, preferably the Dps protein precipitates the nucleic acids already at the concentration of at least 70 μg/μL. The formation of the complex can be measured by gel-shift electrophoresis and the precipitation of the complex can be measured spectroscopically: for example by dynamic light scattering (DLS) technique or by turbidity measurement. In one embodiment the term “Dps protein” encompasses the Dps-like protein. In another embodiment, the Dps protein does not include the Dps-like protein (the items in the summary already reflect the two options).

TABLE 1
Provides amino acid and nucleotide sequence information of an
exemplary Dps protein, such as Dps protein NP_415333.1. The table
includes an amino acid sequence of the protein known under the
accession number NP_415333.1, indicated under SEQ ID NO: 1, and
the nucleotide sequence of the corresponding genome DNA gene
encoding the protein in Escherichia Coli str. K-12 substr.
MG1655, indicated as SEQ ID NO: 2.

| | |
|---|---|
| Amino acid sequence | |
| SEQ ID NO: 1 | mstaklvksk atnllytrnd vsdsekkatv ellnrqviqf |
| | idlslitkqa hwnmrganfi avhemldgfr talidhldtm |
| | aeravqlggv algttqvins ktplksypld |
| | ihnvqdhlkeladryaivan dvrkaigeak dddtadilta |
| | asrdldkflw fiesnie |
| Nucleotide sequence | |
| SEQ ID NO: 2 | atgagtaccg ctaaattagt taaatcaaaa gcgaccaatc |
| | tgctttatac ccgcaacgatgtctccgaca gcgagaaaaa |
| | agcaacagta gagttgctga atcgccaggt |
| | tatccagtttattgatcttt ctttgattac caaacaagcg |
| | cactggaaca tgcgcggcgc taacttcattgccgtacatg |
| | aaatgctgga tggcttccgc accgcactga tcgatcatct |
| | ggataccatggcagaacgtg cagtgcagct gggcggtgta |
| | gctctgggga ccactcaagt tatcaacagcaaaaccccgc |
| | tgaaaagtta cccgctggac atccacaacg ttcaggatca |
| | cctgaaagaactggctgacc gttacgcaat cgtcgctaat |
| | gacgtacgca aagcgattgg cgaagcgaaagatgacgaca |
| | ccgcagatat cctgaccgcc gcgtctcgcg acctggataa |
| | attcctgtggtttatcgagt ctaacatcga ataa |

The term “Dps-like protein” refers to any recombinant or isolated natural protein, or a variant thereof, other than a Dps protein that exhibits functional similarity with a Dps protein of forming a precipitating complex with a nucleic acid as described above. For example Pyrococcus furiosus proteins encoded by the PfDps gene are known to be Dps-like proteins (Ramsay, B. et., J Inorganic Biochem., 100 (2006) 1061-1068.

The term “nucleic acid” or “polynucleotide”, as used interchangeably herein, refer to chains of nucleotides of any length, and include DNA (single- or double-stranded) or RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise unmodified nucleotides. It may also comprise modified nucleotides, such as methylated nucleotides and their analogs. The sequence of nucleotides may be interrupted by non-nucleotide components. A polynucleotide may be further modified after polymerization, such as by conjugation with a labelling component, “caps”, pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), or be modified by substitution of one or more of the naturally occurring nucleotides with an analog. Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S(“thioate”), P(S)S (“dithioate”), (O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The nucleic acid can include, without limitation, genomic DNA, such as host-cell DNA, mitochondrial DNA, host-cell RNA, plasmid DNA, helper virus DNA, helper virus RNA, or fragments thereof. In one embodiment, the nucleic acid can be for example genomic DNA. In another embodiment, optionally after it has been cut by a nuclease, the nucleic acid can be up to about 4.7 kb long, optionally up to 1000 base pairs (bp) long. The polynucleotide can contain between 50 and 200 bp, specifically between 50 and 166 bp, or between 134 and 144 bp. The nucleic acid can be prone to degradation by an endonuclease, preferably a sequence non-specific endonuclease. However, the nucleic acid can also be resistant to an endonuclease. The term “resistant to a nuclease” or “resistant to an endonuclease” in the present context of purifying a composition comprising genetically engineered particles from nucleic acid impurities does not relate to an unwanted nucleic acid being packed in a viral capsid and thus shielded from the nuclease. Instead, it relates to a sequence or a conformation of DNA or RNA, or fragments thereof, that are not cleaved by the nuclease used in a respective purification process, even if a sequence non-specific endonuclease is used. A skilled person will appreciate that a nucleic acid resistant to an nuclease may not always contain the same sequence or conformation, but will depend on the nuclease used in the purification process. The term “resistant to a non-specific endonuclease” relates to the remaining nucleic acids after an endonuclease that is sequence non-specific has been used.

The term “impurity” as used herein relates to any component present in a liquid composition comprising genetically engineered particles that is not the desired product, a product-related substance or an intended formulation excipient. In the context of a nucleic acid impurity the term relates to residual nucleic acid constituents of the production cells, such as host cell DNA and/or RNA, and DNA from helper components, such as plasmids or viruses, or fragments thereof. If used in the process, the impurities can comprise DNA containing antibiotic resistance genes from plasmids, helper viruses, wild type vector genome, or fragments thereof. The methods disclosed herein are aimed at removing the impurity to substantially reduce its content or to remove it completely.

As used herein, “substantially pure” refers to a composition which contains not more than 10 ng residual nucleic acid impurities calculated based on a single therapeutic dose and a median nucleic acid size is of 200 bp or lower. In one embodiment, the measure of substantially pure applies to residual DNA in a composition. In one embodiment, the composition contains not more than 5 ng residual nucleic acids, preferably not more than 2 ng residual nucleic acids.

As used herein “autologous” means that cells, a cell line, or population of cells used for treating patients are originating from said patient or from a Human Leucocyte Antigen (HLA) compatible donor.

As used herein “allogeneic” means that cells or population of cells used for treating patients are not originating from said patient but from a donor.

As used herein, the meaning of an “a” or of a singular noun refers also to plural and includes that of a plural noun. A singular term, unless otherwise indicated, can also carry the meaning of its plural form.

A “subject” as used herein is a mammal, more preferably, a subject is a human. In one embodiment the subject is a patient.

As used herein, “vector” means a construct, which is capable of delivering, and, preferably, expressing, a gene or a polynucleotide sequence of interest in a host cell. The vector can also deliver a gene or a polynucleotide to a host cell in vivo. Examples of vectors include, but are not limited to, a bacterial cell, an eukaryotic cell, a viruses, plasmid, cosmid, phage, DNA or RNA expression vectors associated with cationic condensing agents, DNA or RNA expression vectors encapsulated in a liposome or an extracellular vesicle. In one embodiment the vector is an AAV. In another embodiment a vector is a mammalian cell comprising a polypeptide comprising a chimeric antigen receptor (CAR), wherein the CAR comprises an amino acid modification in the (i) hinge domain, (ii) transmembrane domain, and/or (iii) intracellular domain, wherein the amino acid modification modulates the activity of a CART cell.

As used herein, “pharmaceutical acceptable excipient” includes any material which, when combined with a genetically engineered particle, allows the particle to retain biological activity. The pharmaceutically acceptable excipient is generally non-reactive with the subject's immune system. Examples include, but are not limited to, any of the standard pharmaceutical excipients such as solvents, buffers, pH modifiers, diluents, e.g. phosphate buffered saline solution, water, emulsions such as oil/water emulsion, and various types of wetting agents, tonicity adjusting agents, preservatives, stabilizers, bulking agents, lyoprotectants, colorants, or the like. Compositions comprising such carriers are formulated by well known conventional methods (see, for example, Remington's Pharmaceutical Sciences, 18th edition, A. Gennaro, ed., Mack Publishing Co., Easton, Pa., 1990; and Remington, The Science and Practice of Pharmacy 21st Ed. Mack Publishing, 2005, or Sougata Pramanick, Pharma Times, 2013 45(3), 65-77).

The term “a pharmaceutical composition” refers to a composition comprising genetically engineered particles and a pharmaceutically acceptable excipient that is suitable for administration to a subject, preferably in a method of treatment. Depending on the type of genetically engineered particles formulated in a pharmaceutical composition, intended route of administration, and/or pre-administration steps (such as reconstitution, dissolving, diluting, mixing with pharmaceutically acceptable excipients, or the like), the pharmaceutical composition may be formulated as a solid, liquid or semi-solid composition. For example, a pharmaceutical composition may be a suspension of autologous cells that can be made ready for infusion to a subject. In another embodiment, the pharmaceutical composition can be a bulk or a single dose of AAV vectors in a buffer. A pharmaceutical composition can be prepared in a manner known to a skilled person, for example by means of various conventional steps like mixing, dissolving, stabilizing, lyophilizing, drying, compression, granulating, coating, or other fabrication techniques ready apparent to those skilled in the art. A pharmaceutical composition can comprise a single dose of genetically engineered particles, although that dose may not need to constitute in itself a therapeutically effective amount, since the necessary therapeutically effective amount may be reached by administration of a plurality of doses.

In one embodiment, “purification” or “purifying” can be replaced by “separation”, “isolation” or “removal”. This can be particularly suitable for example in a situation where the nucleic acids removed from a liquid composition comprising genetically engineered particles represent the most valuable part of the composition. For example, this can be applied where the expression product of interest of host cells are in fact naked nucleic acids, which can then be in turn separated from the suspension with the help of a Dps protein.

The term “therapy” or being “therapeutically effective” refers to the treatment of a subject afflicted with, or at risk of contracting or suffering a recurrence of, a disease by a method comprising inducing, enhancing, suppressing or otherwise modifying a cause or a symptom of the disease. In a preferred embodiment, the therapy relates to inducing, enhancing, suppressing or otherwise modifying a cause of the disease.

### Production and Purification of a Polynucleotide-Binding Protein

Production of the Dps protein can be achieved by employing well known methods in the art on how to clone, express and purify a recombinant protein. The methods will generally entail preparing cDNA, selection of a suitable vector, host organism and the like. In relation to vector further variants are possible in relation to a promotor, affinity marker, selection tags and the like. An organism of choice can be Escherichia coli. Production of a protein from E. coli has been extensively discussed by Germán L. Rosano and Eduardo A. Ceccarel in Front Microbiol. 2014; 5: 172. Further source of information on protein production and purification methods can be obtained in Nat Methods. 2008 February; 5(2): 135-146. Exemplary methods relating to Dps, including those of preparing Dps mutant strains and Dps variants can be found in Journal Bacteriology, 2013, doi: 10.1128/JB.00059-13; Journal Bacteriology, October 2015, Vol 197, 19, 3206-15 and Journal of Biological Chemistry, 1999, Vol 274, 46, 33105-13. Those documents contain also other general teaching on Dps proteins.

### Purification

Once a liquid composition comprising genetically engineered particles is mixed with a Dps protein or a Dps-like protein, preferably a Dps protein, and the precipitating complex comprising the nucleic acid impurities and the Dps protein or the Dps-like protein is formed, the complex can be separated from the rest of the composition comprising genetically engineered particles. The methods applied to remove the precipitated complex will be known to a skilled person and can exploit physical or chemical differences between the precipitated complex of the protein-nucleic acid and the wanted genetically engineered particles. For example, the methods used can be, without being limited to, skimming, separation funnel separation, filtration, ultrafiltration, mixer-settler separation, centrifugation, ultracentrifugation, chromatography, or a combination thereof. Optionally, a Dps protein or a Dps-like protein, preferably a Dps protein, can be added to the liquid composition comprising genetically engineered particles more than once. For example, the Dps protein or the the Dps-like protein, preferably the Dps protein can be used to precipitate nucleic acid impurities immediately after the first harvest and optionally a filtration. Then, they can be used again (or for the first time) at any later stage, e.g. before, between or after chromatography or diafiltration. To further clean the media of nucleic acid impurities, the Dps protein (whenever a Dps protein is mentioned, it also applies to a Dps-like protein; although the Dps protein is preferred) can be used in combination with with a nuclease. Various types of nucleases exist. Where removal of nucleic acids is desired, usually endonucleases which are generally sequence non-specific are utilized. Depending of the purification setup, the Dps protein can be added to the liquid composition before the composition has been treated with a nuclease, or after. Again, the sample can be treated with a nuclease more than once, with introducing further purification steps between the treatments. In one embodiment, there need not be any additional purification step between adding a Dps protein and treatment with a nuclease, particularly endonuclease. That means that once the Dps protein has complexed nucleic acids, nuclease can be added to the composition, or vice versa.—the Dps protein is mixed in the liquid composition after nuclease has already cleaved nucleic acids to a certain extent. In any event, due to its indiscriminate nature a Dps protein captures nucleic acids of all sequences of different lengths. As such, the tool is advantageous in that it removes nuclease-resistant nucleic acids. Nuclease-resistant relates to nucleic acids that do not get cleaved by a nuclease, i.e. those that are left in a pharmaceutical composition as an impurity. A very elegant way of removing precipitated complex of a Dps protein and nucleic acids from the remainder of genetically engineered particles is by filtration. Filtration can entail normal flow filtration, dead-end filtration, tangential flow filtration, diafiltration, ultrafiltration, or a combination thereof. Another option is to centrifuge the liquid composition and to exploit different buoyancy characteristics of the precipitated crystals and the genetically engineered particles in a solution or suspension. The precipitated complex (crystals) can be removed also by using chromatography techniques. The basic principles of optional chromatography methods are described in Coscun O., North Clin Istanbul 2016; 3(2):156-60. The chromatography can comprises for example affinity column chromatography, hydroxyapatite chromatography, and/or ion-exchange chromatography. Those methods can be applied for example when manufacturing and processing AAV particles (see Robert et al, Biotechnol. J. 2017, 12, 1600193). Ion-exchange chromatography may be particulary relevant in the preparation of AAV particles. While those methods can be used to remove the precipitated Dps-nucleic acid particles, those can also be included as part of the downstream processing of genetically engineered particles with the aim to cumulatively reach sufficient purity to eventually formulate a pharmaceutical composition comprising genetically engineered particles. In general, the chromatography methods will rely on differences between genetically engineered particles and the precipitated complexes or other impurities, namely in size, affinity, solubility, or the like in different types of matrices or stationary phases. Given that the methods often use virus vectors to facilitate transduction and delivery of DNA during preparation of genetically engineered particles, as well as during treatment, it is important to remove the residual virus nucleic acids. Therefore, in one embodiment, the nucleic acid impurities comprise virus DNA, or a fragment thereof. In another embodiment, the nucleic acid impurity comprise mammalian DNA, or a fragment thereof. The biggest disadvantage or the issue of the presently known purification methods, including the use of nucleases, is that still substantial amount of residual nucleic acid impurities remain in the feedstream and consequently in a final pharmaceutical composition. Therefore, one advantage of the present invention is that it removes also nucleic acids that are resistant to a nuclease. Depending on the selection of genetically engineered particles, e.g. CAR-T cell or virus particles (such as AAV), production yield and the purification steps employed, the final product volume of the final pharmaceutical composition compared to the initial harvest of the engineered particles or some intermittent purification steps can be reduced by a factor of 100- to 500-fold. However, before the final volume is reduced, large quantities of buffers can be used in the process to wash, dilute, or to assist execution of, for example, filter or chromatography methods. The Dps-protein can be efficiently produced in large quantities in bacteria and is thus perfectly suitable for use in larger volumes, such as for example up to 1000 L. Clearly, the amount of protein required to purify larger volumes will expectedly be bigger than the amount for use in smaller volumes. Therefore, a skilled person will know during which step in the overall purification process it is most advantageous to add the Dps protein, with the aim also to reduce the amount of overall buffers needed to purify the genetically engineered particles. The present methods are suitable for use in practically any volume. Generally the volume will be limited by the size of a vessel or a reactor (e.g. bioreactor). The volumes for example can be between about 5 ml and about 500 L, or up to about 100 L, about 10 L, or about 500 mL. In one embodiment, the volume of a composition treated by a Dps protein is between about 500 mL and about 25 L, or between about 500 mL and about 10 L, or between about 500 mL and about 5 L. The volume of the composition can clearly also be smaller, such as about 2 mL, 3 ml, about 5 mL. The benefit of the present invention is that the Dps also shields the nucleic acid impurities in a complex and as such can later used for analysis, for example as to their identity or source of impurities, or their quantity. This advantageous aspect remains available in a situation when a nuclease is added after the Dps protein has been added to the composition comprising genetically engineered particles. Such stepwise approach allows to capture and remove intact impurities and have them available for analysis. In addition, the treatment with a nuclease does not harm the nucleic acids already packed with a Dps protein. However, this is true only to a certain size. Namely, we have done an experiment and the results suggest that a Dps protein was able to successfully protect low molecular weight DNA (sizes below 400 bp) from degradation. On the other hand, higher molecular weight DNA fragments (from genomic DNA contamination), were not effectively protected by Dps protein. The shielding effect was tested in a sample that was left for 9 days at room temperature.

In any event, adding both a Dps protein and a nuclease, preferably not simultaneous, but at different time points or steps in the overall purification process, leads to improved purity of a composition comprising genetically engineered particles and pharmaceutical composition prepared therefrom—namely the improved purity compared to existing methods. The pharmaceutical composition obtained by the methods described herein can be used as a medicine, specifically for a treatment or prevention of a disease.

Therefore, a Dps protein can be used as a purifying agent. A Dps protein can be used in methods disclosed herein. Mixtures comprising genetically engineered particles and a Dps protein contain less free floating nucleic acids, and thus less interfering or contaminating nucleic acids. This holds true for a liquid composition comprising a genetically engineered particles and a Dps protein, as well as for a pharmaceutical composition prepared therefrom, or in alternative, when a pharmaceutical composition itself comprises a Dps protein. Once the composition comprising genetically engineered particles or a pharmaceutical composition is treated with a Dps protein, the level of nucleic acid impurities is deemed lower compared to the levels before the treatment.

Another embodiment of the present invention is a method to assay nucleic acid impurities in a liquid composition comprising genetically engineered particles or a pharmaceutical composition comprising genetically engineered particles, the method comprising the steps of:
(i) adding a Dps protein or a Dps-like protein to the composition or the pharmaceutical composition comprising genetically engineered particles,
(ii) precipitating the residual nucleic acid impurities with the Dps protein or the Dps-like protein,
iii) collecting the precipitated nucleic acid impurities, and
(iv) assaying the nucleic acid impurities.
In one embodiment, the method to assay nucleic acid impurities is used to assay residual nucleic acid impurities. The term “assay” or “assaying” is used herein to refer to an act of identifying, screening, probing or determining by using any conventional means. To assay means herein to optionally subject a sample, for example the removed complex of Dps protein-nucleic acids, to an analytical assay protocol, which may include, for example, purification, transformation or further preparation of the respective sample to bring it in a form suitable for analysis; and testing and/or measuring. These steps can be performed in any order and the final protocol can be optimized. Such downstream handling of the separated complex can be combined with any suitable separation, purification or analysis method, including purification with magnetic particle technology. Release of the polynucleotide from the complex can be achieved by hydrolysis of the Dps protein, for example by proteinase, such as proteinase A or K. The nucleic acid can be released by resuspending the complex in a solution comprising generally increased concentration of Mg2+ ions. Increased molar concentrations of Mg2+ ions in a solution abolish Dps—nucleic acid interaction. The Mg2+ ion concentration should be adjusted based on the protein used. Another option to release nucleic acids from the complex is to disrupt the structure of the complex by precipitating the protein with ethanol or phenol, preferably phenol. Released nucleic acids can be resuspended in a low-salt solution, including water. The complex, or nucleic acid separated from the complex can be further purified. Purification methods will be readily known to a person skilled in the art. The available purification methods that can be applied according to the present disclosure are for example utilizing bi-phase system phenol:chloroform:isoamyl alcohol (e.g. in ratio 25:24:1), which denaturates the Dps protein while forces nucleic acid into water phase. Purification methods can include also re-precipitation of nucleic acids, chromatography, or using the advantage of ability of silica to bind nucleic acids in the form of nucleic acid-binding magnetic beads, where silica coated paramagnetic beads are added to the samples to bind nucleic acid (e.g. Agencourt AM Pure beads from Beckman Coulter), spin columns with silica-coated membrane (e.g. from QIAGEN) or any other silica-coated material. With regards to silica coated magnetic beads, the mixture of beads and nucleic acid are immobilized on magnets and washed to remove remaining protein and contaminants. Removal of residual binding solution is executed with a second wash solution and finally thnucleic acid is eluted in a low-salt buffer. Various suitable nucleic acid purification kits are commercially available. The obtained and optionally purified polynucleotide can be subjected to downstream applications, such as PCR and sequencing. For example, the quantity of the residual nucleic acid impurities thus separated can be determined. The residual nucleic acid impurities can also be assayed for a presence of a specific impurity, such as a marker indicating contamination (e.g. by external virus, bacteria, mycoplasma, fungi, yeast or the like, that is otherwise not used in preparation of genetically engineered particles). The residual nucleic acid impurities thus separated can also be assayed to determine their identity, or source. The assaying techniques and means used to assay the nucleic acid impurities can be PCR-based techniques. They can comprise using polymerase chain reaction (PCR), digital PCR, droplet digital PCR, sequencing, next generation sequencing, Northern blot analysis, Southern blot analysis, reverse transcription-polymerase chain reaction (RT-PCR), TaqMan-based assay, high-density oligonucleotide SNP array, restriction fragment length polymorphism (RFLP) assay, dynamic allele-specific hybridization, primer extension assay, oligonucleotide ligase assay, analysis of single strand conformation polymorphism, temperature gradient gel electrophoresis (TGGE), denaturing high performance liquid chromatography, high resolution melting analysis, DNA mismatch-binding protein assay, capillary electrophoresis, immunoassay, ELISA, mass spectroscopy, or the like. Available technologies to interrogate nucleic acid are described also in Table 1 of Nature Reviews, April 2017, Vol 17, 223-38. In addition, the nucleic acids retrieved by the process as described herein can be assayed for epigenetic modifications, such as DNA methylation or other alterations that do not relate to changes in DNA sequence.

### EXAMPLES

### Preparation of a Dps Protein

Dps protein was expressed and purified according to Karas et al. (Karas V O, Westerlaken I, Meyer A S. 2013. Application of an in vitro DNA protection assay to visualize stress mediation properties of the Dps protein. J Vis Exp 75:50390). Briefly, protease-deficient strain of E. coli (such as BL21(DE3) pLysS) was transformed with a pET vector (such as pET17) into which the Dps protein-encoding gene sequence has been cloned. Cells were grown at 37° C. with shaking at 250 rpm to an optical density at 600 nm of 0.4 to 0.6, and expression of Dps was induced by addition of isopropyl-D-thiogalactopyranoside (IPTG) to a concentration of 0.3 mM, and incubated at 37° C. for 3-4 hr while shaking. Cells were harvested by centrifugation at 6,000×g for 15 min and pellet resuspended in 7.5 ml of DEAE buffer A (50 mM HEPES-KOH, pH 7.5, 100 mM NaCl, 0.1 mM EDTA) per L of induced cell culture. To prevent degradation of over expressed Dps, a mixture of protease inhibitors was added. Cells were lysed using French press and clarified of insoluble particles by centrifugation at 30,000×g for 35 min at 4° C. after clarification, the lysate was purified with a combination of ion-exchange column chromatography (e.g. DEAE-Sepharose as AEX and e.g. SP Sepharose as CEX chromatography media) and ammonium sulfate precipitation. The concentration of purified Dps protein (NP_415333.1) samples was determined by measuring the absorbance at 280 nm, with a molar extinction coefficient of 15,470 M⁻¹ cm⁻¹ for the Dps monomer. The Dps protein concentration in the final storage buffer (50 mM HEPES-KOH, pH 7.5, 50 mM NaCl) was ˜7 mg/mL or 10 mg/mL (as indicated in the experiments below). The following experiments were performed by using the prepared Dps protein as a representative Dps protein.

### Example 1. Precipitation of DNA from Solution using Dps Protein

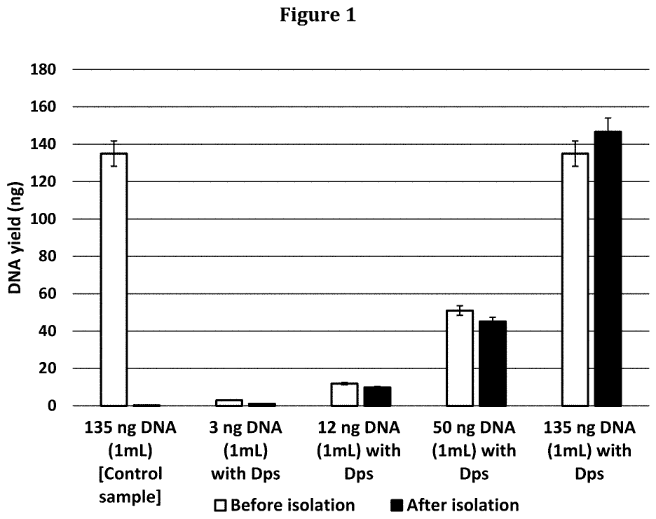

To demonstrate the functionality of Dps protein for precipitating nucleic acids from a solution, a proof-of-concept experiment was performed. To this end, 1 mL solutions with various DNA concentrations were prepared by suspending DNA standard (NEB, 100bp ladder) in nuclease-free water (NFW; Nuclease-free Water (Ambion®)). Additionally, 10 μL of the prepared Dps protein (7 mg/mL) was added and samples were incubated at 33° C. for 30 min. After incubation, samples were centrifuged at 21000×g for 15 min at 4° C. and supernatant was discarded. Pelleted Dps-DNA complex (i.e. biocrystals) was resuspended in 50 μL water. Proteinase K was added and samples were incubated at 56° C. for 10 min in order to release Dps from DNA. Subsequently, DNA was purified with DNA-binding magnetic beads (i.e. 90 μL magnetic beads were used, following two consecutive washes with 80% ethanol). DNA was eluted from magnetic beads with 50 μL water and DNA concentration was measured. All DNA measurements were performed fluorometrically. The isolation process using Dps protein resulted in more than 90% recovery of initial DNA amount in all Dps containing samples (FIG. 1). Recovery was not dependent on the initial DNA concentration in the sample. On the other hand, DNA solution without added Dps protein (control sample) resulted in the complete loss of DNA. In summary, this experiment confirms that DNA can be precipitated from solution using Dps.

### Example 2. Dps Binds to DNA without Apparent Sequence and Size Specificity

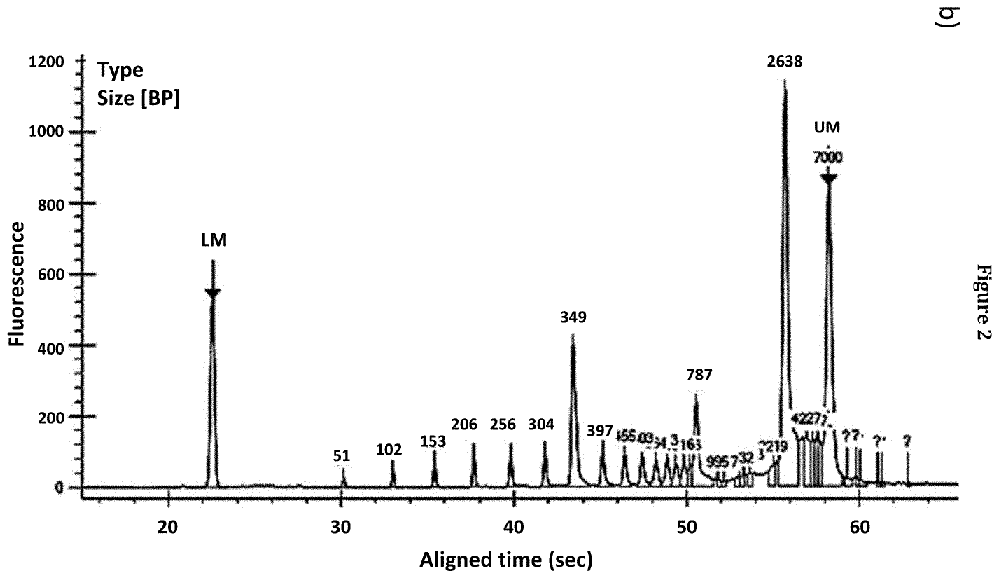

To demonstrate the functionality of Dps protein for separating nucleic acids from a solution, a proof-of-concept experiment was performed. To this end, 5 mL DNA solution was prepared by suspending DNA standard (50 bp DNA ladder; Invitrogen:10416-014) in nuclease-free water (NFW; Nuclease-free Water, Ambion®). Additionally, 100 μL of Dps protein (10 mg/mL) was added and samples were incubated at 33° C. for 30 min. After incubation, samples were centrifuged at 20 000×g for 15 min at 4° C. and supernatant was discarded. Pelleted Dps-DNA complex was resuspended in 500 μL water. Proteinase K was added and sample was incubated at 56° C. for 10 min in order to release Dps from DNA. Subsequently, DNA was purified using the QIAamp DNA Blood Mini Kit (Qiagen) following manufacturer guidelines and purified DNA was analysed on capillary electrophoresis in order to determine the size of the captured DNA. As shown in FIG. 2, size distribution and intensities of DNA fragments before and after Dps-based extraction are comparable, confirming that Dps captures DNA of all sizes and independently of the nucleotide sequence.

### Example 3. Removal of a Genomic DNA from a Solution using Dps Protein

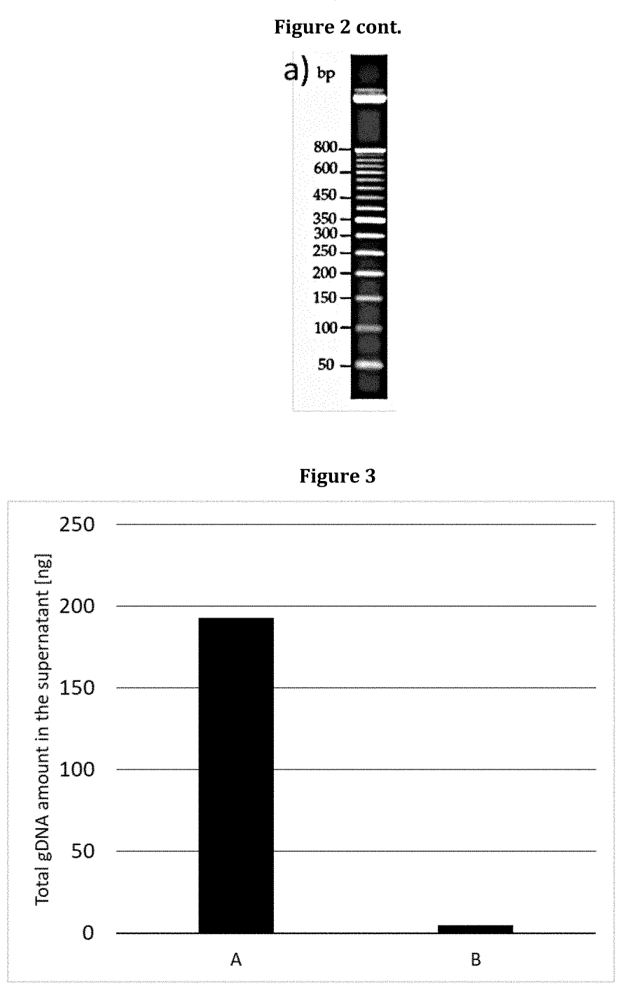

To demonstrate the functionality of Dps protein for separating genomic DNA (gDNA) from a solution, a proof-of-concept experiment was performed. To this end, a cell suspension of 4×10⁶ viable mammalian cells in 400 μL nuclease-free water (NFW) was prepared and vortexed vigorously for 1 minute in order to mechanically disrupt the cells and release genomic DNA. Next, two 50 uL aliquots of cell lysate (A and B) were prepared and 150 μL NFW was added to each aliquot. In addition, 20 μL of the Dps protein (10 mg/mL) was added to sample B and both samples were then incubated on ice. After 10 min incubation samples were centrifuged at 20 000×g for 10 min and the amount of gDNA in the supernatant was measured fluorometrically using the Qubit system (Invitrogen). The use of the Dps protein resulted in more than 40-fold decrease of gDNA amount in the supernatant (4.6 ng) when compared to gDNA amount in the supernatant without the use of Dps (193 ng) (FIG. 3).

### Example 4. Accumulation of Nucleic Acids from a Larger Volume for Reliable Detection and Quantification

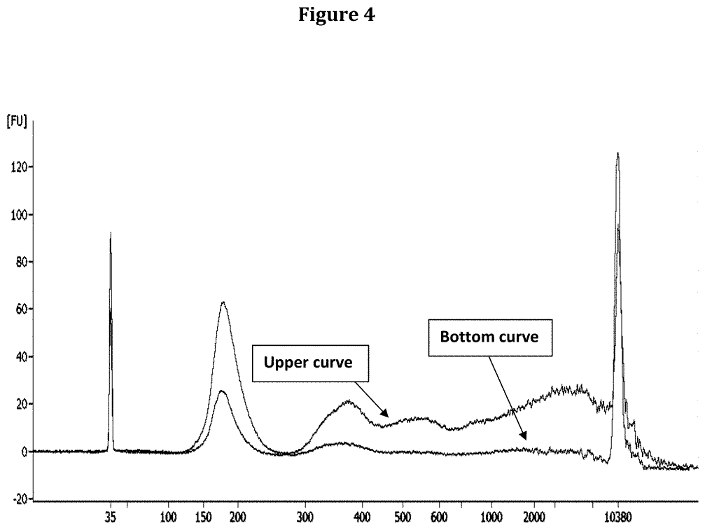

A proof-of-concept experiment was carried out to demonstrate that the cfDNA accumulated from a larger volume with a Dps protein is compatible with existing and commercially available DNA isolation kits for further purification. A biological sample comprising cells and an unknown concentration of nucleic acids was provided and centrifuged first at 3000×g for 10 min at 4° C. to remove cells. After centrifugation, the supernatant sample was transferred into a fresh centrifuge tube and centrifuged again at 16000×g for 10 min at 4° C. to remove residual cells and cell debris. Then, two aliquots were prepared from the second supernatant. The cfDNA was extracted from one aliquot by using solely the QIAamp MinElute ccfDNA Mini Kit (QIAGEN) and from the second by a combination of the QIAamp MinElute ccfDNA Mini Kit after Dps had been added to the second aliquot. Using solely the QIAamp MinElute ccfDNA Mini Kit, cfDNA was extracted from 1 mL of the sample following manufacturer's instructions. The extraction of cfDNA with Dps protein in combination with QIAamp MinElute ccfDNA Mini kit was performed as follows. First, 7.5 mL of the sample was diluted in 30 ml of nuclease-free water and 300 μL of the Dps protein (7 mg/mL) was added. After incubation by gentle mixing at RT for 80 minutes, the solution was centrifuged at 16.000×g for 15 min at 4° C. Supernatant was carefully decanted and pellet was resuspended in 1000 μL nuclease-free water. Obtained Dps-cfDNA solution was further purified with QIAamp MinElute ccfDNA Mini kit as per manufacturer's instructions. Then, the DNA from both extractions was visualized using Bioanalyzer 2100 (Agilent Technologies). Results show that applying Dps and extracting DNA with the help of Dps and with commercially available cfDNA purification kit (in this case QIAamp MinElute ccfDNA Mini kit by QIAGEN) allows extraction of higher-yields of cfDNA from larger sample volumes using the equal amount of reagents provided in the commercial kit (FIG. 4). It should be noted that the QIAamp MinElute ccfDNA Mini kit (without the use of Dps) allows extraction of cfDNA from only up to 4 mL of a sample. This results clearly show that a Dps protein can easily be added to or combined with available extraction or purification methods. A Dps protein allows, for example, capturing nucleic acid impurities from larger volumes before they are further tested by other known processes, or stabilizes them before being processed.

### Example 5. Removal of a Host-Cell DNA from a Suspension of AAV Particles using Dps Protein

To demonstrate the functionality of a Dps protein for separating host cell DNA (hcDNA) from a solution of AAV particles, a proof-of-concept experiment was performed. To this end, AAV2 viral vector solution (ATCC® VR-1616™; Lock et al. Human Gene Ther. October 21(10):1273-1285, 2010) was mixed with HEK293 genomic DNA (considered as hcDNA) or with 1× Phosphate Buffered Saline (PBS; to serve as control sample) as follows: for sample “A”, 50 μL of AAV2 was mixed with 450 μL of PBS, and for sample “B” 33 μL of AAV2 was mixed with 297 μL (approximately 3 ng) of HEK293 DNA (to yield approximate concentration of 6 ng/mL). Three 100 μL aliquots of each sample (A and B) were prepared. To the first aliquots of A and B (1A, 1B) 30 μL of PBS was added, to the second aliquots of A and B (2A, 2B) 30 μL of the Dps protein (10 mg/mL) was added, and to the third aliquots of A and B (3A, 3B) DNase reaction mixture, comprising of 15 μL PBS, 2 μL DNase I enzyme (Ambion), 13 μL 10× DNase I buffer (Ambion), was added. Samples 1A and 1B were incubated at room temperature for 30 min. Samples 2A and 2B were first incubated at 4° C. for 30 min and then centrifuged at 4° C. and 16.000 g for 15 min. Supernatant was removed and transferred to a new tube after centrifugation from 2A and 2B (yielding samples 2AS and 2BS, “S” stands for supernatant) and the pellet was resuspended with 100 μL of PBS (yielding samples 2AP and 2BP; “P” stands for pellet). Samples 3A and 3B were incubated at 37° C. for 30 min. Next, 100 μL of each resulting sample was used for automated extraction of residual DNA using PrepSEQ™ Residual DNA Sample Preparation Kit (Thermo Fisher Scientific, A27335) resulting in 200 μL of eluted DNA for each of the eight samples (see Table 2).

TABLE 2
Summary of samples for DNA extraction:

Group

| Sample | 1 | 2 (supernatant) | 2 (pellet) | 3 |
|---|---|---|---|---|
| A | 1A | 2AS | 2AP | 3A |
| B | 1B | 2BS | 2BP | 3B |

Residual DNA was quantified using resDNASEQ™ Human Residual DNA Quantitation Kit (ThermoFischerScientific, A26366) according to the instruction manual for reaction preparation. Extracted DNA of each sample was diluted 3× prior qPCR reaction and 10 μL of the samples was added to the reaction. Results show that for samples A (diluted with PBS), hcDNA was present below the limit of quantification (LOQ; 0.3 pg/reaction). For samples B the hcDNA could be quantified in B2 (untreated sample) at 8.96 ng/mL (SD=0.84) and in B2P (pellet after Dps treatment) at 7.44 ng/mL (SD=2.11). For samples 2BS (supernatant after Dps treatment) and 3B (DNase reaction) hcDNA was present below the LOQ. These results show that hcDNA was efficiently removed from the composition comprising AAV2 by a Dps protein, since the hCDNA was quantified in the pellet and not in supernatant. Based on further measurements of the samples with different dilutions of the hcDNA concentration, it was evident that a Dps protein can remove at least 100-fold increased amount of nucleic acids from the composition.

1. A method for purifying a liquid composition comprising genetically engineered particles from nucleic acid impurities comprising the steps of (i) adding a Dps protein or a Dps-like protein to the liquid composition comprising genetically engineered particles, (ii) precipitating a complex comprising the nucleic acid impurities and the Dps protein or the Dps-like protein and (iii) removing the precipitated complex comprising the nucleic acid impurities and Dps protein from the composition.

2. The method for purifying a liquid composition comprising genetically engineered particles from nucleic acid impurities according to claim 1, wherein the method further comprises a step of skimming, separation funnel separation, filtration, ultrafiltration, mixer-settler separation, centrifugation, ultracentrifugation, chromatography, use of a magnetic particle technology, utilization of magnetic properties of the Dps protein or the Dps-like protein, sterilization and/or treatment of the liquid composition comprising genetically engineered particles with a nuclease, or a combination thereof.

3. The method for purifying a liquid composition comprising genetically engineered particles from nucleic acid impurities according to claim 1, wherein the steps as defined in claim 1 are repeated more than once, optionally at different stages of the method for purifying the liquid composition comprising genetically engineered particles.

4. The method for purifying a liquid composition comprising genetically engineered particles from nucleic acid impurities according to claim 1, where the method further comprises adding a nuclease to the liquid composition comprising genetically engineered particles.

5. (canceled)

6. (canceled)

7. (canceled)

8. (canceled)

9. The method for purifying a liquid composition comprising genetically engineered particles from nucleic acid impurities according to claim 1, wherein the nucleic acid impurity is resistant to a nuclease.

10. The method for purifying a liquid composition comprising genetically engineered particles from nucleic acid impurities according to claim 1, wherein the concentration of Mg2+ ions in the liquid composition comprising genetically engineered particles during the step of precipitating the complex comprising the nucleic acid impurities and the Dps protein or the Dps-like protein is not more than 7.5 mM.

11. The method for purifying a liquid composition comprising genetically engineered particles from nucleic acid impurities according to claim 1, wherein the volume of the liquid composition comprising genetically engineered particles is from about 5 mL and up to about 1000 L.

12. (canceled)

13. The method for purifying a liquid composition comprising genetically engineered particles from nucleic acid impurities according to claim 1, further preparing a pharmaceutical composition comprising the genetically engineered particles.

14. (canceled)

15. A liquid composition comprising genetically engineered particles obtained by a method according to claim 1.

16. (canceled)

17. (canceled)

18. (canceled)

19. (canceled)

20. (canceled)

21. (canceled)

22. (canceled)

23. (canceled)

24. (canceled)

25. (canceled)

26. The method for purifying a liquid composition comprising genetically engineered particles from nucleic acid impurities according to claim 1, wherein the genetically engineered particles are a recombinant protein, prokaryotic cell, eukaryotic cell, a virus, a bacteriophage, a nanostructured particle, an extracellular vesicle or a liposome, or a combination thereof.
